(19) United States
(12) Patent Application Publication
(10) Pub. No.: US 2025/0084363 A1
(43) Pub. Date: Mar. 13, 2025

(54) AUTOMATED CULTURING APPARATUS AND AUTOMATED CULTURING METHOD

(71) Applicants: CANON KABUSHIKI KAISHA, Tokyo (JP); Canon Medical Systems Corporation, Otawara-shi (JP)

(72) Inventors: Shuhei IKEDA, Yokohama (JP); Takaaki KAWANO, Chigasaki (JP); Tomoaki YAMAKI, Yokohama (JP)

(73) Assignees: CANON KABUSHIKI KAISHA, Tokyo (JP); Canon Medical Systems Corporation, Otawara-shi (JP)

(21) Appl. No.: 18/823,799

(22) Filed: Sep. 4, 2024

(30) Foreign Application Priority Data
Sep. 8, 2023 (JP) 2023-146363

(51) Int. Cl. C12M 1/36; C12M 1/00; C12M 1/34; C12M 3/00

(52) U.S. Cl. CPC C12M 41/48; C12M 23/44; C12M 29/06; C12M 41/14; C12M 41/46

(57) ABSTRACT

According to one embodiment, an automated culturing apparatus includes a plurality of incubators, an imaging unit and processing circuitry. The plurality of incubators is configured to store one or more culture containers in which a cell or a biological tissue is cultured. The imaging unit is configured to capture an image of the cell or the biological tissue in the culture containers in the incubators. The processing circuitry is configured to control delivery and discharge of a cell suspension and a medium for the culture containers stored in each of the incubators, and control a culturing condition of the cell or the biological tissue independently for each incubator.

### CROSS-REFERENCE TO RELATED APPLICATIONS

This application is based upon and claims the benefit of priority from Japanese Patent Application No. 2023-146363, filed Sep. 8, 2023, the entire contents of which are incorporated herein by reference.

### FIELD

Embodiments described herein relate generally to an automated culturing apparatus and an automated culturing method.

### BACKGROUND

The development of regenerative medicine has drawn attention to the culturing of cells such as induced pluripotent stem cells (iPS cells). The reproducibility of the cell culturing often varies depending on the pipetting technique, resulting in an unfavorable situation where there is a variation in the reproducibility among operators. Thus, the use of an automated culturing apparatus that enables cell culturing without human intervention has been considered. However, since different culturing conditions cannot be set for each culture container, it is difficult to set optimal culturing conditions according to the degree of growth of individual cells.

### BRIEF DESCRIPTION OF THE DRAWINGS

FIG. 1 is a block diagram of an automated culturing apparatus according to an embodiment.

FIG. 2 is a diagram showing a first example of a configuration of an imaging unit according to the embodiment.

FIG. 3 is a diagram showing a second example of a configuration of an imaging unit according to the embodiment.

FIG. 4 is a flowchart showing a culturing process performed by the automated culturing apparatus according to the embodiment.

### DETAILED DESCRIPTION

In general, according to one embodiment, an automated culturing apparatus includes a plurality of incubators, an imaging unit and processing circuitry. The plurality of incubators is configured to store one or more culture containers in which a cell or a biological tissue is cultured. The imaging unit is configured to capture an image of the cell or the biological tissue in the culture containers in the incubators. The processing circuitry is configured to control delivery and discharge of a cell suspension and a medium for the culture containers stored in each of the incubators, and control a culturing condition of the cell or the biological tissue independently for each incubator.

Hereinafter, an automated culturing apparatus and an automated culturing method according to an embodiment will be described with reference to the accompanying drawings. In the embodiment described below, elements assigned with the same reference symbols perform the same operations, and repeat descriptions will be omitted as appropriate.

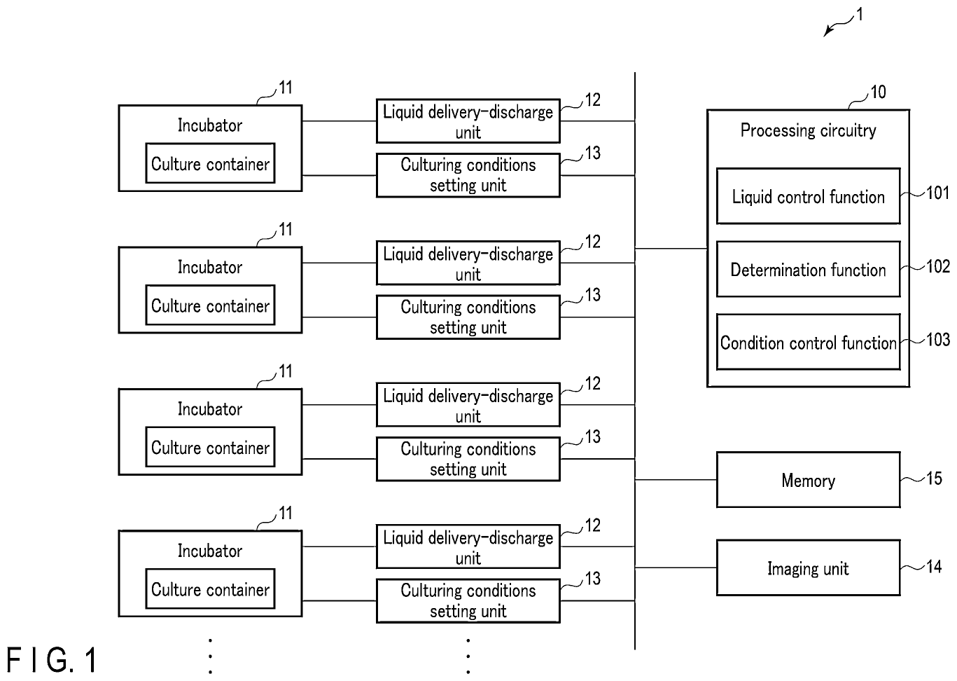

A conceptual view of an automated culturing apparatus according to an embodiment will be described with reference to the block diagram of FIG. 1.

An automated culturing apparatus 1 according to an embodiment includes processing circuitry 10, a plurality of incubators 11, a plurality of liquid delivery-discharge units 12, a plurality of culturing conditions setting units 13, an imaging unit 14, and a memory 15. The incubators 11, the liquid delivery-discharge units 12, and the culturing conditions setting units 13 are associated with each other on a one-on-one basis. That is, in the example shown in FIG. 1, the liquid delivery-discharge units 12 and the culturing conditions setting units 13 are connected to the four incubators 11, respectively. The embodiment is not limited thereto, and it suffices that there are multiple incubators 11, and that multiple incubators 11 are arranged according to the specifications of the automated culturing apparatus 1.

The processing circuitry 10 is a processor and includes a liquid control function 101, a determination function 102, and a condition control function 103.

The liquid control function 101 controls, via the liquid delivery-discharge units 12, the delivery and discharge of a cell suspension or a medium, the amount of liquid delivery, the interval of liquid delivery, etc., for a culture container which is stored in each of the incubators 11 and in which cell or biological tissue culturing is performed.

Hereinafter, a case of culturing cells will be explained, but a culturing process can be implemented in the same manner even in the case of culturing a biological tissue. Examples of the cells include not only various cultured cell lines such as CHO cells derived from Chinese hamster ovaries, mouse connective tissue L929 cells, mouse skeletal myoblasts (C2C12 cells), normal diploid fibroblasts derived from human fetal lungs (TIG-3 cells), cells derived from human fetal kidneys (HEK293 cells), A549 cells derived from human alveolar basal epithelial adenocarcinomas, and HeLa cells derived from human cervical cancers, but also epithelial cells and endothelial cells constituting various tissues and organs in a living body, skeletal muscle cells, smooth muscle cells and cardiac muscle cells showing contractility, neuron cells, glial cells and fibroblasts constituting the nervous system, hepatic parenchymal cells, hepatic non-parenchymal cells, and fat cells participating in the metabolism of the living body, various stem cells such as iPS cells, embryonic stem(ES) cells, embryonic germ (EG) cells, embryonic carcinoma (EC) cells, mesenchymal stem cells, hepatic stem cells, pancreatic stem cells, skin stem cells, muscle stem cells and germ stem cells, precursor cells of various tissues, and cells differentiation-induced therefrom.

The culture container is assumed to be, for example, a plastic container called a dish, but may be any container that is chemically stable and allows culturing of desired cells.

The determination function 102 determines the state of cell growth. Specifically, the determination function 102 determines, as the state of cell growth, the degree of cell growth such as the number of cell colonies, the sizes of the colonies, and the confluence. The confluence is a ratio of cells covering the bottom surface of the culture container.

The condition control function 103 executes control to set the conditions of culturing the cells or the living tissue independently for each incubator based on, for example, an image captured by the imaging unit 14.

The incubator 11 is a constant-temperature device for maintaining the temperature, humidity, and gas composition such as the concentration of carbon dioxide and the concentration of oxygen constant. A culture container such as a dish can be stored in the incubator 11. It is assumed that a single culture container is stored in one incubator 11, but a plurality of culture containers may be stored in one incubator 11. The incubator 11 has a structure that allows the imaging unit 14, which is described later, to capture an image of the culture container inside the incubator 11. For example, a transparent acrylic panel may be provided to a part of the housing of the incubator 11 so that an image of the culture container inside the incubator 11 can be captured. Alternatively, the incubator 11 may be provided with an open-close mechanism such as a window or a lid so as to have a structure that allows the imaging unit 14 to capture an image while a part of the housing is open.

The liquid delivery-discharge unit 12 is connected to the incubator 11 via a tube member such as a tube. The liquid delivery-discharge unit 12 delivers a cell suspension, a medium, or the like to the culture container inside the incubator 11 through a pipe. In the process of replacing a medium, the liquid delivery-discharge unit 12 suctions a medium from the culture container through, for example, a pipetting unit (not shown) and a tube that are arranged inside the incubator 11, discharges the medium to the outside, and delivers a new medium to the culture container. Also, in the process of collecting cells, the liquid delivery-discharge unit 12 collects cells from the culture container through a pipetting unit and a tube.

The culturing conditions setting unit 13 is connected to the incubator 11 via a tube member such as a tube, a heat plate, or the like. The culturing conditions setting unit 13 sets the temperature, humidity, and gas composition such as carbon dioxide or oxygen inside the incubator 11 according to the control instruction from the condition control function 103.

The imaging unit 14 is, for example, a phase-contrast microscope, and captures an image of the cells in the culture container inside each incubator 11. Thus, a user can observe the cells inside the culture container and check the state of cell growth such as the number of colonies, the sizes of the colonies, and the confluence. The imaging unit 14 is not limited to a phase-contrast microscope and may be an optical microscope such as a bright field microscope or a fluorescent microscope, an electronic microscope, or an optical camera.

The memory 15 is, for example, a non-volatile memory such as an HDD (hard disk drive) or an SSD (solid state drive), and holds an image of the inside of the culture container captured by the imaging unit 14 and data of, for example, the state of the growth of the cells in the culture container stored in each incubator 11.

Although not shown, the automated culturing apparatus 1 may further include a display for displaying, for example, a captured image and data relating to imaging conditions, and an input-output interface for outputting the data to the outside and for acquiring input from a user.

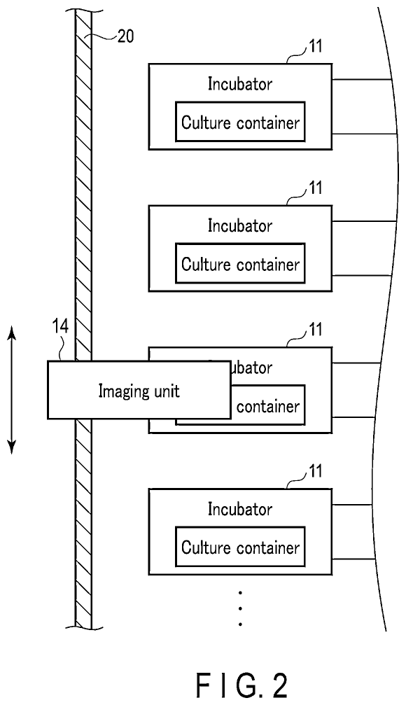

Next, a first example of a configuration of the imaging unit 14 according to the embodiment will be described using FIG. 2.

FIG. 2 is a conceptual view showing an arrangement relationship between the imaging unit 14 and the incubators 11.

The imaging unit 14 is movably arranged in a one-dimensional direction along a first shaft 20 extending along the direction in which the incubators 11 are arranged. For example, the imaging unit 14 may be connected to the first shaft 20 via a linear bushing (not shown), etc., and guided in linear motion along the extending direction of the first shaft 20. The first shaft 20 is arranged above the plane where the incubators 11 are arranged so that the imaging unit 14 does not collide with the incubators 11. Thus, the imaging unit 14 can capture individual images of the inside of the culture containers in the respective incubators 11.

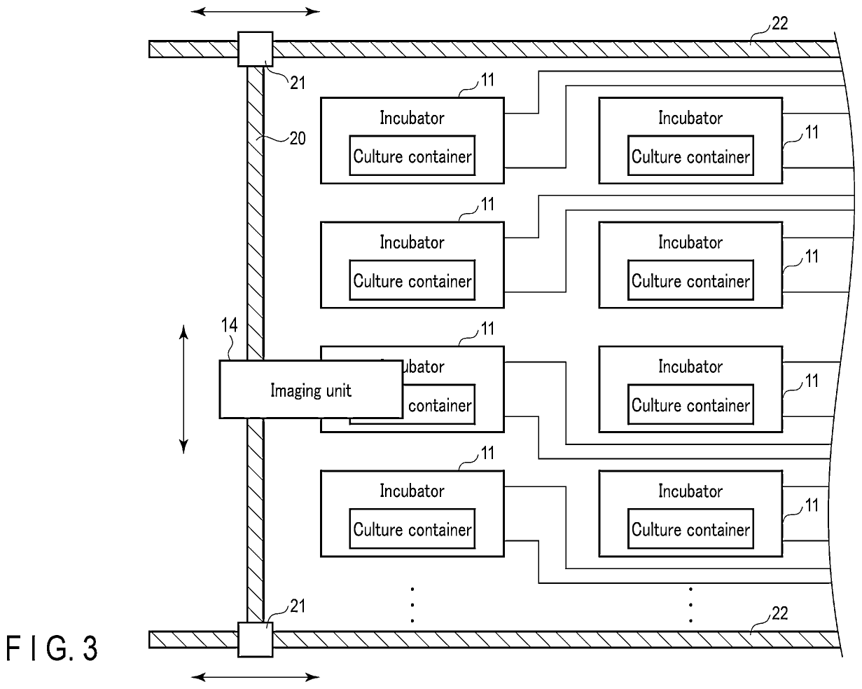

Next, a second example of a configuration of the imaging unit 14 according to the embodiment will be described using FIG. 3.

While FIG. 2 shows an example in which the incubators 11 are arranged in a line, FIG. 3 shows an example of a configuration of the imaging unit 14 when the incubators 11 are arranged two-dimensionally.

Here, the state in which eight incubators 11 are arranged two-dimensionally is shown. The first shaft 20 is connected to two second shafts 22 via a mover 21. The mover 21 may be formed to have a structure that enables linear-motion guide represented by, for example, a linear bushing.

When the incubators 11 are viewed from the upper side, the second shaft 22 extends in a direction orthogonal to the extending direction of the first shaft 20 in the area outside the area in which the incubators 11 are arranged and is connected to the mover 21. The first shaft 20, the mover 21, and the second shaft 22 are arranged above the plane where the incubators 11 are arranged so that the imaging unit 14 does not collide with the incubators 11.

Thus, the imaging unit 14 can be freely moved over a two-dimensional plane parallel to the plane where the incubators 11 are arranged, which makes it possible to capture individual images of the culture containers in the respective incubators 11 even if the incubators 11 are arranged two-dimensionally. The embodiment is not limited to the second configuration example shown in FIG. 3; it is also possible to arrange a three-dimensionally movable arm above the incubators 11, connect the imaging unit 14 to a distal end of the arm, and capture individual images of the culture containers.

Also, in the examples shown in FIGS. 2 and 3, the imaging unit 14 captures images from above the respective incubators 11; however, the embodiment is not limited thereto. The imaging unit 14 may be arranged below the incubators 11 and capture images of the culture containers in the incubators 11 from the lower side. In this case, the incubators 11 may be formed to have, as the structure of the incubators 11, a structure that allows imaging from below the housing.

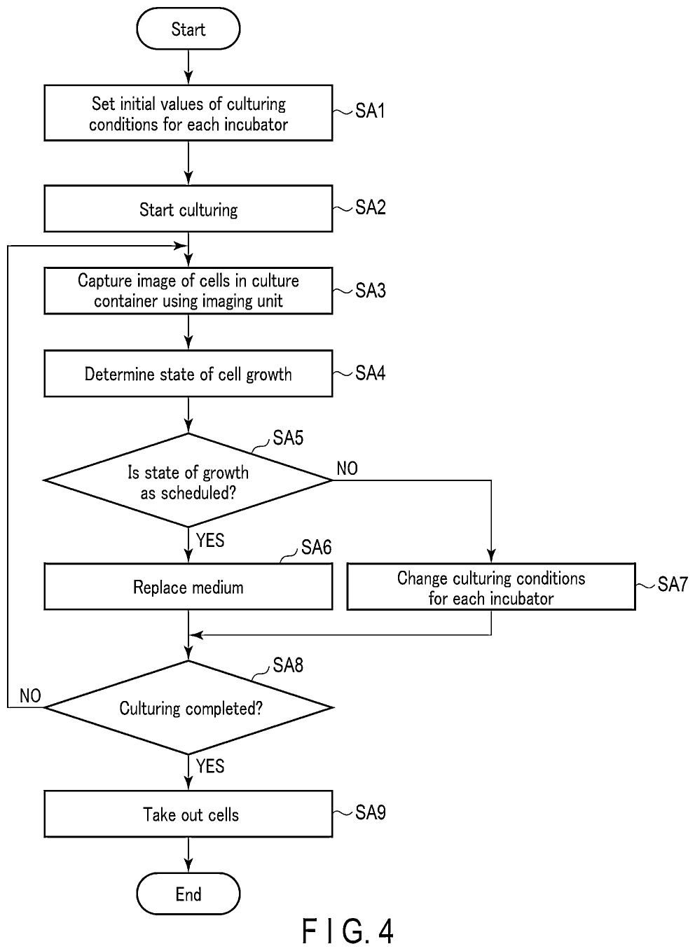

Next, a culturing process performed by the automated culturing apparatus 1 according to the embodiment will be described with reference to the flowchart of FIG. 4.

In step SA1, the processing circuitry 10 sets initial values of the culturing conditions independently for each incubator 11 by implementing the condition control function 103. Specifically, after one or more culture containers in which cells are seeded are stored in each incubator 11, the processing circuitry 10 implements the condition control function 103 to set the culturing conditions, specifically, the temperature, humidity, and concentration of carbon dioxide via the culturing conditions setting units 13. For example, a certain incubator 11 is set to a temperature of 37° C., a humidity of 95%, and a carbon dioxide concentration of 5%, and another incubator 11 is set to a different temperature of 36.5° C., a humidity of 95%, and a carbon dioxide concentration of 5%. In this manner, different culturing conditions may be set to the respective incubators 11. The initial values of the culturing conditions may be set to differ among the respective incubators 11 based on empirically determined values according to the type of cells cultured.

The same culturing conditions can also be set for the multiple incubators 11. For example, the same initial values may be set for every two incubators 11.

In step SA2, the automated culturing apparatus 1 starts culturing based on the set initial values of the culturing conditions.

In step SA3, the imaging unit 14 captures an image of a culture container in each incubator 11 at predetermined intervals and acquires the images of the culture containers. For example, with the structures shown in FIG. 2 or FIG. 3, the imaging unit 14 captures images of the cells in the culture containers while going around the incubators 11. The predetermined intervals may be, for example, once daily at a fixed time, multiple time intervals within a day such as 8 o'clock, 12 o'clock, 16 o'clock, etc., or may be any interval, such as every two days, that allows proper observation of the cell growth.

In step SA4, by implementing the determination function 102, the processing circuitry 10 determines the state of cell growth based on the images captured by the imaging unit 14. For example, with the determination function 102, the processing circuitry 10 may calculate the number of cell colonies, the sizes of the colonies, and the confluence by analyzing the images.

In step SA5, by implementing the determination function 102, the processing circuitry 10 determines whether or not the state of cell growth follows the culturing schedule. For example, if it is immediately after the start of cell culturing, it is determined that the state of cell growth follows the culturing schedule if cell adhesion to the culture container and cell division can be confirmed from the image. Also, if certain days have passed after the start of culturing, it may be determined that the state of cell growth follows the culturing schedule if at least one of the number of cell colonies, the sizes of the colonies, or the confluence calculated by the image analysis is equal to or above a growth prediction value as a threshold at the specified schedule.

If the state of cell growth is as scheduled, the process proceeds to step SA6, and if the state of cell growth is not as scheduled, the process proceeds to step SA7.

In step SA6, by implementing the liquid control function 101, the processing circuitry 10 controls the liquid delivery-discharge unit 12 to replace the medium. The liquid delivery-discharge unit 12 suctions (collects) the medium in the culture container in the incubator 11 and delivers a new medium to the culture container. It is assumed that the medium replacement is performed without taking out the culture container to the outside of the incubator 11. Specifically, the liquid delivery-discharge unit 12 may be connected to a pipette for collecting a medium and a pipette for adding a medium in the incubator 11, each of the pipettes may be operated through the control executed by the liquid control function 101, and collection of the medium in the culture container and addition of a medium to the culture container may be performed.

Also, by implementing the liquid control function 101, the processing circuitry 10 may control the amount of medium delivered based on the state of cell growth. For example, if the cell growth is fast, a process of increasing the amount of liquid delivery may be performed so that the amount of medium is increased. Also, by repeating the process from step SA3 to step SA6, the processing circuitry 10, with the liquid control function 101, controls the intervals of delivering a medium based on the state of cell growth.

In step SA7, by implementing the condition control function 103, the processing circuitry 10 controls the culturing conditions setting units 13 to change the culturing conditions (temperature, humidity, gas composition) for each incubator 11 that stores a culture container of cells not growing as scheduled. For example, the culturing conditions set for an incubator 11 showing the fastest cell growth state are set as the culturing conditions of other incubators 11. Specifically, by implementing the condition control function 103, the processing circuitry 10 controls the culturing conditions setting units 13 to apply, for example, the temperature, humidity, and gas composition set for an incubator 11 that stores a culture container having the largest size of colonies to other incubators 11 as well. Thus, even though the degree of growth varies according to the individual differences among the cells, a management that accelerates the growth of the cells stored in each incubator 11 can be expected.

On the other hand, if the cell growth is faster than scheduled, the culturing conditions set for an incubator 11 showing the slowest cell growth state may be set as the culturing conditions of other incubators 11. Thus, although there are individual differences among the cells, as in the case of accelerating the growth, a management that decelerates the growth of the cells stored in each incubator 11 can be expected, allowing for application to, for example, the needs of culturing high-quality cells using a small number of colonies.

In step SA8, by implementing the determination function 102, the processing circuitry 10 determines whether or not the culturing is completed based on the images captured by the imaging unit 14. For example, it may be determined that the culturing is completed if it can be determined based on the images that the cell-occupied area ratio in the medium reaches the so-called subconfluence, which is approximately 70 to 80%.

If the culturing is completed, the process proceeds to step SA9; if the culturing is not completed, the process returns to step SA3, and the same process is repeated.

In step SA9, the culture containers are removed from the incubators 11 and the cells are collected. Through the above process, the culturing process performed by the automated culturing apparatus 1 is completed.

In the culturing process shown in FIG. 4, the medium is replaced if the state of cell growth is as scheduled; however, the embodiment is not limited to this example. The processing circuitry 10 may perform the medium replacement via the liquid delivery-discharge units 12 at any timing such as when there is a user instruction by implementing the liquid control function 101.

The present embodiment assumes that there is one imaging unit 14, and that an image of the culture container stored in each incubator 11 is captured by moving the imaging unit 14; however, the imaging unit 14 may be fixedly installed exclusively in each incubator 11. That is, if there are eight incubators 11, eight imaging units 14 may be installed.

According to the embodiment described above, the automated culturing apparatus has multiple incubators that store culture containers, and images of the culture containers in the respective incubators are captured as the imaging unit moves. Control can be executed to set the culturing conditions independently for each incubator.

Since this enables automated culturing using multiple incubators, it is possible to suppress variation in culturing caused by operators. Further, since an image of each incubator can be captured by the imaging unit, it is possible to execute independent control while optimizing the culturing conditions according to the degree of cell growth. Therefore, highly efficient culturing can be realized.

The terminology “processor” used in the above description refers to, for example, circuitry such as a CPU (central processing unit), a GPU (graphics processing unit), an ASIC (application specific integrated circuit), a programmable logic device (such as a simple programmable logic device (SPLD), a complex programmable logic device (CPLD), and a field programmable gate array (FPGA)), etc. If the processor is, for example, a CPU, the processor implements the functions by reading and executing programs stored in storage circuitry. On the other hand, if the processor is an ASIC, for example, its functions are directly incorporated into the circuitry of the processor as logic circuitry, instead of a program being stored in the storage circuitry. Each processor of the present embodiment is not limited to being configured as single circuitry; multiple sets of independent circuitry may be integrated into a single processor that implements its functions. Furthermore, the functions may be implemented by a single processor into which multiple components shown in the drawings are incorporated. That is, efficient and highly precise inspection can be implemented.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An automated culturing apparatus comprising:
a plurality of incubators configured to store one or more culture containers in which a cell or a biological tissue is cultured;
an imaging unit configured to capture an image of the cell or the biological tissue in the culture containers in the incubators; and
processing circuitry configured to
control delivery and discharge of a cell suspension and a medium for the culture containers stored in each of the incubators, and
control a culturing condition of the cell or the biological tissue independently for each incubator.

2. The automated culturing apparatus according to claim 1, wherein the imaging unit captures an image of the cell or the biological tissue in the one or more culture containers in each of the incubators by moving in a one-dimensional direction along a direction in which the incubators are arranged.

3. The automated culturing apparatus according to claim 1, wherein the imaging unit captures an image of the cell or the biological tissue in the one or more culture containers in each of the incubators by moving over a two-dimensional plane parallel to a face on which the incubators are arranged.

4. The automated culturing apparatus according to claim 1, wherein the processing circuitry is further configured to:
determine a growth state of the cell or the biological tissue in the culture containers; and
control at least one of temperature, humidity, or gas composition in each of the incubators based on the growth state.

5. The automated culturing apparatus according to claim 1, wherein the processing circuitry is further configured to:
determine a growth state of the cell or the biological tissue in the culture containers; and
control at least one of an amount of the medium delivered or an interval of delivering the medium based on the growth state.

6. The automated culturing apparatus according to claim 1, wherein the processing circuitry is configured to set a culturing condition set for an incubator showing a fastest growth state of the cell or the biological tissue in a culture container as a culturing condition of other incubators.

7. The automated culturing apparatus according to claim 1, wherein the processing circuitry is configured to set a culturing condition set for an incubator showing a slowest growth state of the cell or the biological tissue in a culture container as a culturing condition of other incubators.

8. The automated culturing apparatus according to claim 4, wherein the growth state is determined based on at least one of a number of colonies, a size of the colonies, or a confluence of the cell or the biological tissue.

9. The automated culturing apparatus according to claim 1, wherein each of the incubators stores a single culture container.

10. An automated culturing method comprising:
controlling delivery and discharge of a cell suspension and a medium for one or more culture containers which are stored in each of incubators and in which a cell or a biological tissue is cultured;
capturing an image of the cell or the biological tissue in the culture containers in the incubators; and
controlling a culturing condition of the cell or the biological tissue independently for each incubator.

11. The automated culturing method according to claim 10, wherein the capturing the image of the cell or the biological tissue in the one or more culture containers in each of the incubators is performed by moving an imaging unit in a one-dimensional direction along a direction in which the incubators are arranged.

12. The automated culturing method according to claim 10, wherein the capturing the image of the cell or the biological tissue in the one or more culture containers in each of the incubators by moving an imaging unit over a two-dimensional plane parallel to a face on which the incubators are arranged.

13. The automated culturing method according to claim 10, further comprising:
determining a growth state of the cell or the biological tissue in the culture containers; and
performing a control of at least one of temperature, humidity, or gas composition in each of the incubators based on the growth state.

14. The automated culturing method according to claim 10, further comprising:
determining a growth state of the cell or the biological tissue in the culture containers; and
performing a control of at least one of an amount of the medium delivered or an interval of delivering the medium based on the growth state.

15. The automated culturing method according to claim 10, further comprising setting a culturing condition set for an incubator showing a fastest growth state of the cell or the biological tissue in a culture container as a culturing condition of other incubators.

16. The automated culturing method according to claim 10, further comprising setting a culturing condition set for an incubator showing a slowest growth state of the cell or the biological tissue in a culture container as a culturing condition of other incubators.

17. The automated culturing method according to claim 13, wherein the growth state is determined based on at least one of a number of colonies, a size of the colonies, or a confluence of the cell or the biological tissue.

18. The automated culturing method according to claim 10, wherein each of the incubators stores a single culture container.
